(19) United States
(12) Patent Application Publication
(10) Pub. No.: US 2019/0055195 A1
(43) Pub. Date: Feb. 21, 2019

(54) Phenyl, bromo-maleimide as a coupling group for conjugation to sulfhydryl group

(71) Applicant: Immunwork Inc., Taipei City (TW)

(72) Inventors: Tse-Wen CHANG, Taipei City (TW); Hsing-Mao CHU, Taipei City (TW)

(73) Assignee: Immunwork Inc., Taipei City (TW)

(21) Appl. No.: 16/029,657

(22) Filed: Jul. 9, 2018

Related U.S. Application Data
(60) Provisional application No. 62/547,121, filed on Aug. 18, 2017.

(51) Int. Cl. C07D 207/456; A61K 31/454; A61K 47/54; A61K 47/68; A61K 47/60

(52) U.S. Cl. CPC C07D 207/456; A61K 31/454; A61K 47/60; A61K 47/6889; A61K 47/545

(57) ABSTRACT

The invention pertains to the creation of two new classes of compounds, referred to as “phenyl, bromo-maleimide acids” and “Phenyl, bromo-maleimide amines”, which are derivatized from the maleimide, C₂H₂(CO)₂NH (2,5-pyrroledione) and have the backbone structure of formula I and II below,

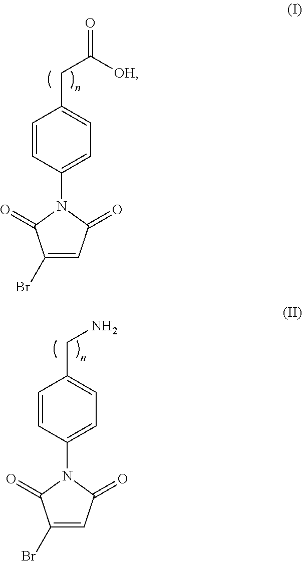

where n is 0-6. In the maleimide ring, a hydrogen in the 3,4-imide (C₂H₂) is substituted by a bromide and the nitrogen is bonded to a phenyl group, which is bonded to a carboxylic acid or amine. The invention also pertains to the use of these two classes of compounds to tag molecules with a Br,Ph-maleimide group or to synthesize hetrobifunctional linkers with a Br,Ph-maleimide group for conjugating various molecules to the sulfhydryl groups of cysteine residues in proteins, peptides, nanoparticles, or cells. Specifically, the Br,Ph-maleimide group has the structure of formula (III):

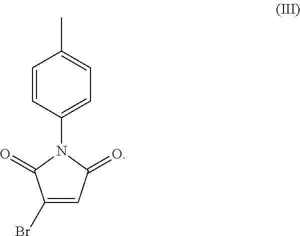

### FIELD OF INVENTION

The invention is in the field of bioconjugation, in which a biological or small organic molecule or a bundled group of such molecules is conjugated to a protein, peptide, nanoparticles, or cells, or a bundled group of such proteins or peptides.

### SUMMARY OF INVENTION

I. Maleimide and Thiol Reaction is Often Applied in Bioconjugation

Maleimide group and sulfhydryl group of cysteine residues can react specifically at around neutral pH. This Michael type of reaction can occur amid other nucleophilic groups such as amino groups of lysine residues. Therefore, among many functional groups that can react with sulfhydryl group, maleimide is used frequently, such as in preparing antibody drug conjugates for use in treating caner cells, wherein the cytotoxic drug molecules are derivatized with a linker and a terminal maleimide group.

It is known that adducts of maleimide and thiol groups can undergo retro reaction or exchange reaction in the presence of other thiol-containing molecules (Baldwin A D and Klick K L. Bioconjug Chem 19:1946-53, 2011). While such retro or exchange reactions may provide a mechanism for slow release, it is often not desirable. In addition, the maleimide and thiol reaction may not proceed sufficiently rapidly to avoid side products or other unwanted effects. It is also known that the succinimide moiety of maleimide and thiol adduct can undergo ring-opening hydrolysis (Scheme 1) and the resultant molecules then become stable (Fontaine S D et al. Bioconjug Chem 26:145-52, 2015) toward cleavage, avoiding retro or exchange reactions.

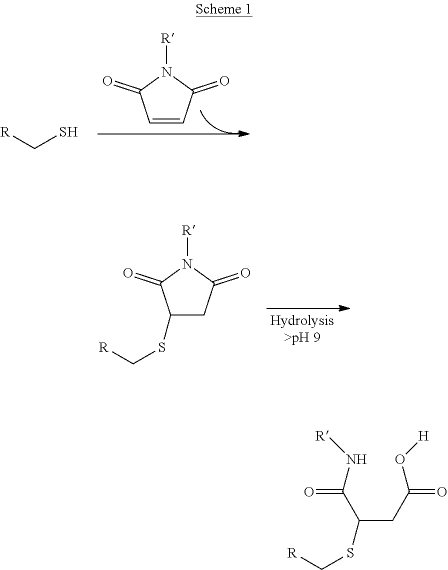

II. Effects of Bromide Substitution at the 3,4-imide and of Phenyl Substitution at the N Position of the 2,5-pyrroledione on the Stability of the Maleimide-Thiol Adducts

The activity of the 3,4-imide as an electrophile is affected by the substitutions at the 3 or 4 positions. Bromide substitution of a hydrogen at the 3 or 4 position increase the pulling of electron toward the bromide, resulting in increased reactivity of the imide as an electrophile, decreased retro activity of the maleimide-thiol adduct, and increased conversion to the hydrolyzed ring-opened stable product (Smith M E et al. Org Biomol Chem 13:7946-9, 2015). In separate studies, a phenyl substitution at the N position has also been shown to result in pulling the electron of the pyrroledione ring toward the phenyl ring, which can increase reactivity of the maleimide with thiol, decrease retro reaction, and increase ring opening hydrolysis and hence more stable adduct (Christie R J et al. J Controlled Release 220:660-70, 2015).

It has not been investigated whether the combination of a bromide substitution at the 3 or 4 position and a phenyl substitution at the N position of maleimide can result in a similar or even higher reactivity of the imide, lesser retro activity, higher ring-opening hydrolysis, and hence higher stability of the adduct than either of the substitutions alone. Compounds and methodologies for modifying molecules or proteins with a maleimide group with bromide and phenyl substitutions have not been available.

III. Synthesis of a Series of Bromo-phenyl-maleimide Acids with 3-bromo and N-phenyl Acid Substitutions in the 2,5-pyrroledione (Br,Ph-maleimide Acids)

Compounds with the structures of formula (I) shown below have not been reported before the present invention:

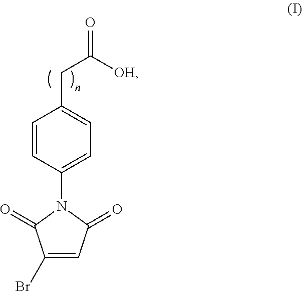

where n =0 to 6.

The above compounds are synthesized by the following reactions shown in Scheme 2. Compounds 1 with n=0, namely, 4-aminobenzoic acid (CAS No. 150-13-0), with n=1, namely, 4-aminophenylacetic acid (CAS No. 1197-55-3), and with n=2, namely, 3-(4-aminophenyl)propionic acid (CAS No. 2393-17-1) and compound 2, bromomaleic anhydride (CAS No. 5926-51-2) are commercially available. Compounds 3 are newly synthesized in this invention disclosure. Compounds 3 are referred to as Br,Ph-maleimide acids.

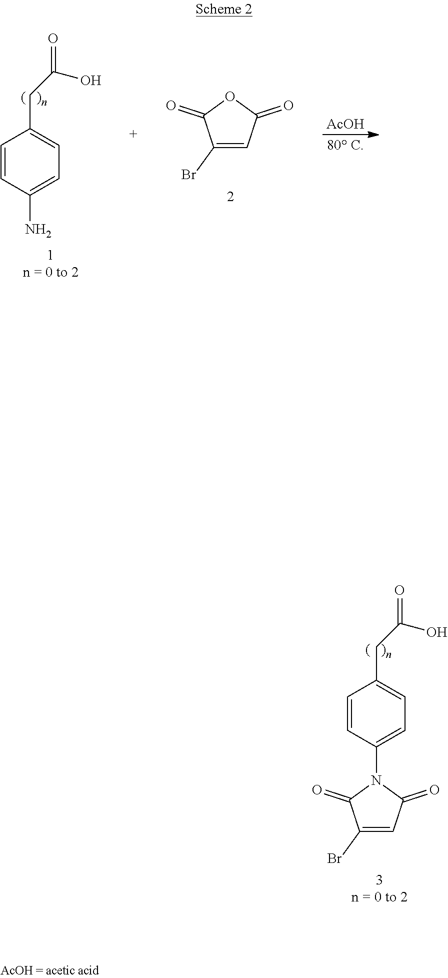

The phenyl ring may also be substituted with electron-pulling groups, such as chloride, fluoride or NO₂. The imide in the maleimide ring may also be substituted with two bromide groups. The phenyl ring and the acid may also contain other linkage.

IV. Synthesis of a Series of Bromo-phenyl-maleimide Amines with 3-bromo and N-phenyl Acid Substitutions in the 2,5-pyrroledione (Br,Ph-maleimide Amines)

Compounds with the structure of formula (II) shown below have not been reported before the present invention:

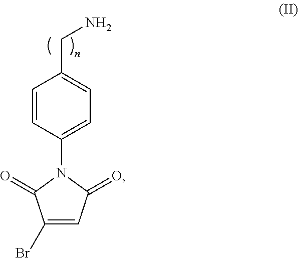

where n =0 to 6.

The above compounds can be synthesized as shown in Scheme 3. Compounds 4 with n=1, namely, 4-aminobenzyl alcohol (CAS No. 623-04-1) and with n=2, namely, 4-aminophenethyl alcohol (CAS No. 104-10-9) are commercially available. Compounds 5, 6, and 7 are newly synthesized in this disclosure. Compounds 7 are referred to as Br,Ph-maleimide amines.

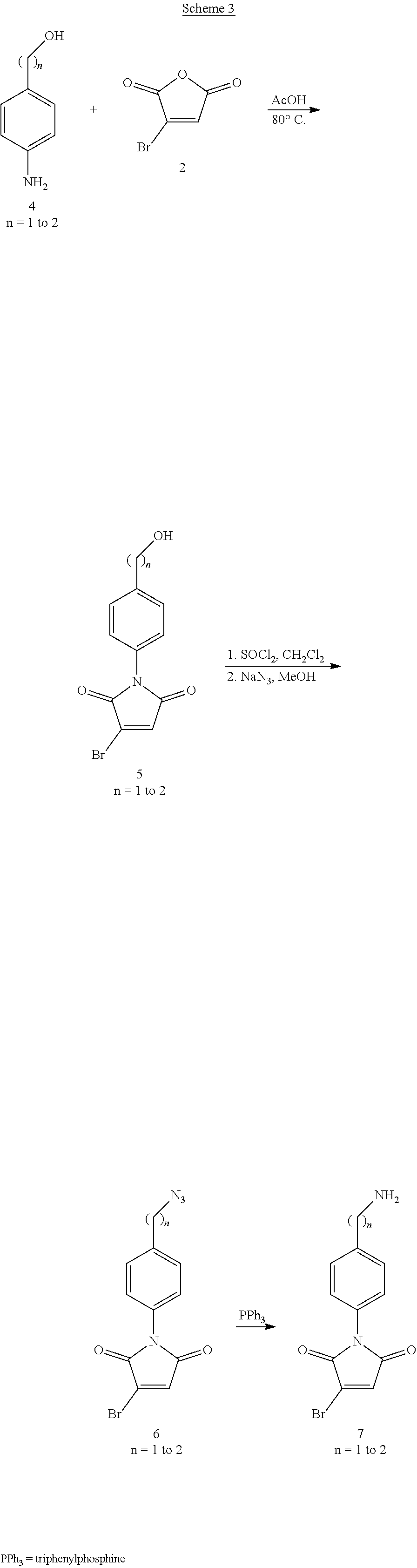

V. Uses of Br,Ph-maleimide Acids and Br,Ph-maleimide Amines

A. Tagging a Molecule with a Br,Ph-maleimide Group

A compound in the Br,Ph-maleimide acids series can be used to react with an amino group in a molecule in a typical amide formation reaction, introducing a 3-bromo, N-phenyl-maleimide (Br,Ph-maleimide) group. Analogously, a compound in the BR,Ph-maleimide amines series can be used to react with a CO₂H group in a molecule in a typical amide formation reaction, introducing a Br,Ph-maleimide group. The tagged molecules can then be conjugated to the thiol group of cysteine residues on antibody molecules, single-chain antibody variable fragments (scFv), single-domain antibody (sdAb), or other proteins. This utility can be applied to small molecular drugs, such as cytotoxic drug molecules, e.g. mertansine (DM1), monomethyl auristane (MMAE), pyrrolobenzodiazepine (PBD), erybulin, lenalidomide, irinotecan; an immunosuppressor, e.g. sirolimus, tacrolimus, everolimus; an immunostimulator, e.g. monophosphoryl lipid A; and an enzyme inhibitor, e.g. thrombin inhibitor, argatroban. If the molecules do not have an amine or CO₂H group, such a group may be introduced by derivatization. An example of derivatizing lenalidomide (compound 8) using a Br-Ph-maleimide acid (compound 9) to produce a Br,Ph-maleimide group-tagged lenalidomide (compound 10) is shown in Scheme 4.

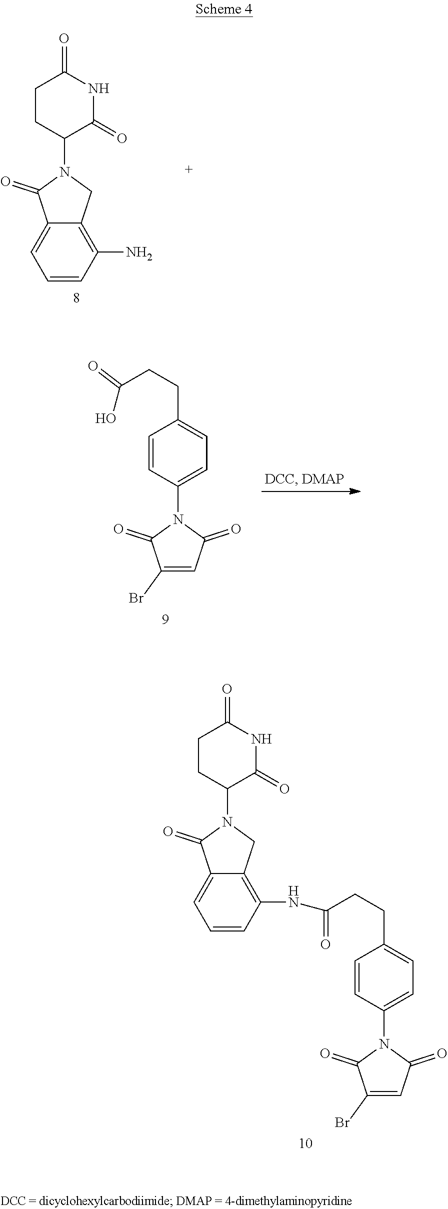 [SMILES: Nc1cccc2c1CN(C1CCC(=O)NC1=O)C2=O ; O=C(O)CCc1ccc(N2C(=O)C=C(Br)C2=O)cc1 ; O=C1CCC(N2Cc3c(NC(=O)CCc4ccc(N5C(=O)C=C(Br)C5=O)cc4)cccc3C2=O)C(=O)N1]

B. Tagging a Heterobifunctional Linker with a Br, Ph-maleimide Group

Using the same amide formation chemistry as described in A, the amine or CO₂H group on one end of a heterobifunctional linker can be tagged with a Br,Ph-maleimide group.

The heterobifunctional linkers have a different coupling group at the other end, such as one used in click chemistry, including alkyne, azide, dibenzocyclooctyne (DBCO), bicyclononyne (BCN), difluorinated cyclooctyne (DIFO), dibenzocyclooctyne (DICO), tetrazine, trans-cyclooctene (TCO), and norbornene, each of which can react with a countering functional group in click chemistry. The chemical nature of the linker between the Br,Ph-maleimide group and the other coupling group is a peptide, polyethylene glycol, or their combination, or other polymeric substances. An example of using a Br,Ph-maleimide acid (compound 11) to modify a heterobifunctional linker with NH₂ at one end and TCO at the other end and PEG in between the functional groups (compound 12) to produce a heterobifunctional linker with Br,Ph-maleimide group at one end and a TCO group at the end (compound 13) is shown in Scheme 5.

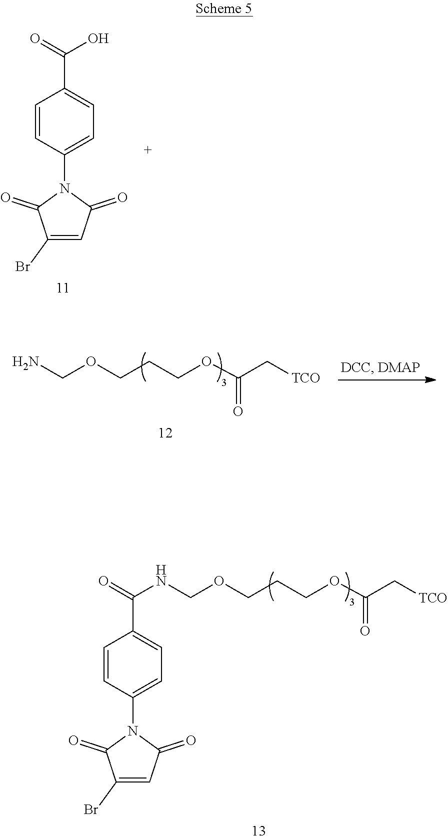

C. Using Heterobifunctional Linkers to Introduce a Functional Group Onto a Protein or a Peptide

A protein, such as albumin, tissue plasminogen activator or its fragments, scFv, sdAb, may already have a sulfhydrl group or genetically engineered to contain an extra linker that contain a cysteine residue(s) or site-specifically mutated to contain a cysteine residue(s). The sulfhydryl group can then be reacted with heterobifunctional linkers with a Br, Ph-maleimide group at one end and a functional group at the other end for click chemistry. An example of using a heterobifunctional linker with Br,Ph-maleimide group at one end and a TCO group at the other end (compound 13) to tag two TCO groups on an IgG molecule, which is engineered to contain two terminal cysteine residues, is shown in Scheme 6.

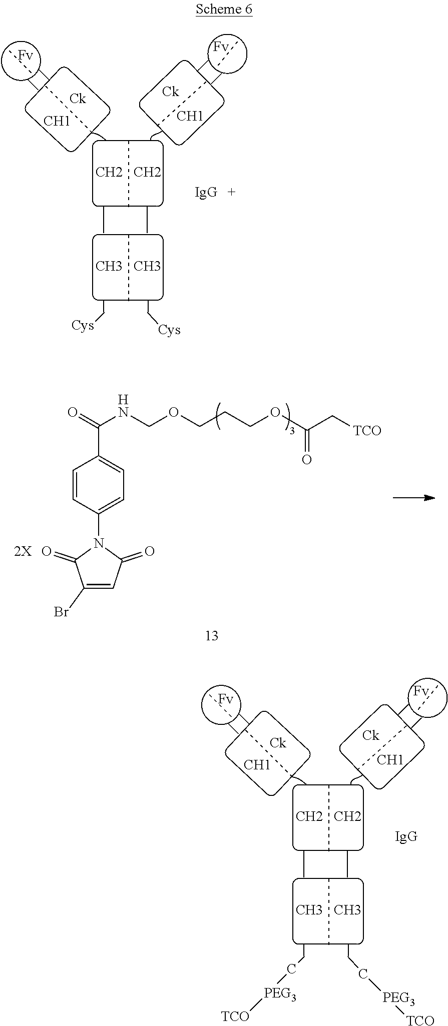

Antibody fragment, scFv, can be engineered to contain a C-terminal linker and a cysteine residue, providing a thiol group for conjugation. An example of using a heterobifunctional linker with a Br,Ph-maleimide group at one end and a TCO group at the other end (Compound 13) to tag an scFv with a TCO group (compound 14) is shown in Scheme 7. In order to prevent compound 14 to react with an additional scFv molecule, cysteine is added to modify and quench the imide on the maleimide ring (compound 15). Compound 15 can then undergo ring-opening hydrolysis to yield stable compound 16.

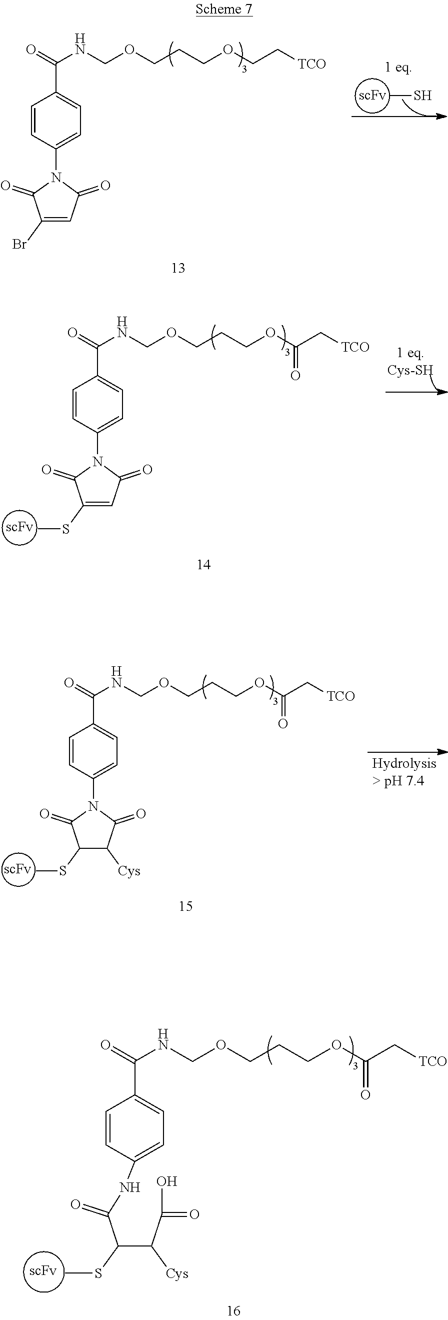

D. Installing a Coupling Arm Onto the Center Core of a Multi-Arm Linker

In some embodiments of multi-arm linkers (US2016/0208020A1), the peptide center core contains a cysteine residue as the base for attaching a coupling arm with a functional group at the far end for click chemistry. The heterobifunctional linkers with a Br,Ph-maleimide group at one end and a functional group at the other end for click chemistry can be applied. An example of using a heterobifunctional linker with Br,Ph-maleimide group at one end and a TCO group at the other end (compound 13) to tag a TCO group on a peptide with cysteine residue is shown in Scheme 8.

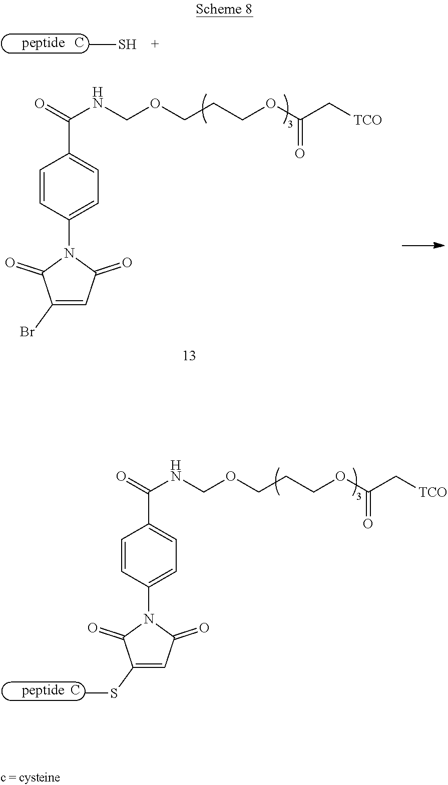

E. Applying Br,Ph-maleimide Group at the Ends of Linking Arms in a Multi-Arm Linker

The linking arms of multi-arm linkers are primarily for conjugating with various targeting or effector elements, which have a sulfhydryl group for coupling. The linking arms can be prepared as peptides or PEG with Br,Ph-maleimide group at the end. An example of using a heterbifunctional linker with Br,Ph-maleimide group at one end and a CO₂H at the other end (compound 17) to modify the NH₂ groups of 3 lysine residues in a TCO-tagged peptide through amide formation is shown in scheme 9.

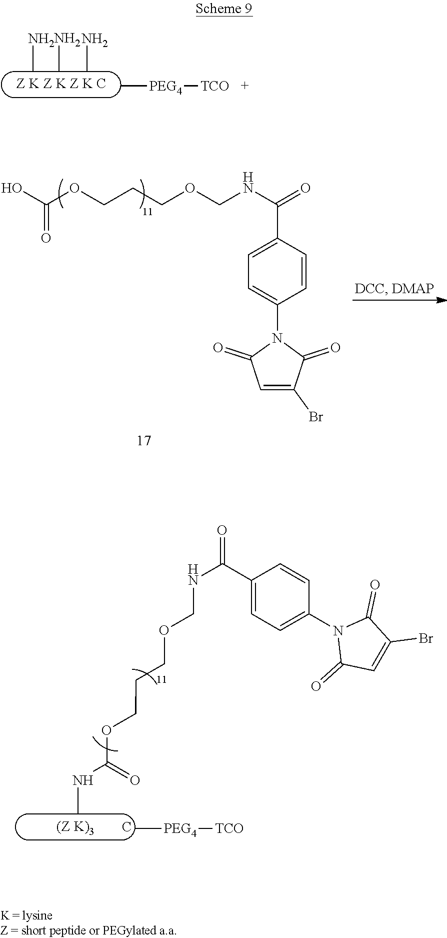

What is claimed:

1. A compound having the structure of formula (I): 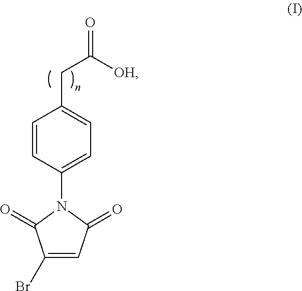
where n=0 to 6.

2. A compound having the structure of formula (II): 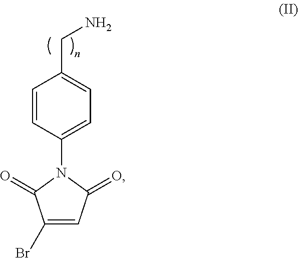
where n=0 to 6.

3. A method of tagging a Br,Ph-maleimide group onto a molecule with an amine group by conjugating with a compound of claim 1, wherein the Br,Ph-maleimide group has the structure of formula (III)

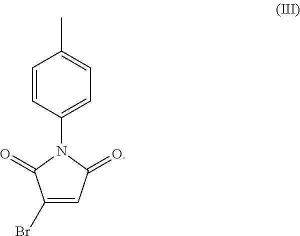

4. A method of tagging a Br,Ph-maleimide group onto a molecule with a CO₂H group by conjugating with a compound of claim 2, wherein the Br,Ph-maleimide group has the structure of formula (III)

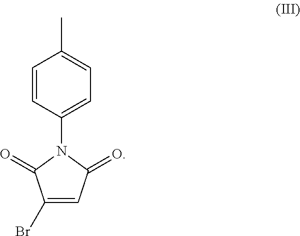

5. The method of claim 3 or 4, in which the molecule is a small molecule with biological activity.

6. The method of claim 5, in which the small molecule is selected from the group consisting of mertansine (DM1), monomethyl auristane (MMAE), pyrrolobenzodiazepine (PBD), erybulin, lenalidomide, irinotecan, sirolimus, tacrolimus, everolimus, monophosphoryl lipid A, and argatroban.

7. The method of claim 3 or 4, in which the molecule is a heterobifunctional linker with amine or CO₂H group at one end and a functional group for click chemistry at the other end.

8. The method of claim 7, in which the functional group for click chemistry is selected from the group consisting of alkyne, azide, dibenzocyclooctyne (DBCO), bicyclononyne (BCN), difluorinated cyclooctyne (DIFO), dibenzocyclooctyne (DICO), tetrazine, trans-cyclooctene (TCO), and norbornene.

9. A method of tagging a functional group for click chemistry to a peptide with a cysteine residue, in which the thiol group is reacted with a heterobifunctional linker with a Br,Ph-maleimide group at one end and a functional group for click chemistry at the other end, wherein the Br,Ph-maleimide group has the structure of formula (III)

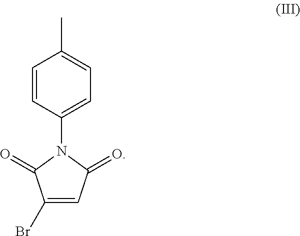

10. The method of claim 9, in which the functional group for click chemistry is selected from the group consisting of azide, alkyne, DBCO, BCN, DIFO, DICO, tetrazine, and TCO.

11. A method of tagging 2 or more CH(Br)CH₂(CO)₂N—C₆H₄-groups onto a peptide with 2 or more lysine residues, in which heterobifunctional linkers with a Br,Ph-maleimide group at one end and CO₂H group at the other end are conjugated to the amine groups of the lysine residues via amide bond formation reaction, wherein the Br,Ph-maleimide group has the structure of formula (III) [chemical structure]
